(19) United States
(12) Patent Application Publication
(10) Pub. No.: US 2026/0139195 A1
(43) Pub. Date: May 21, 2026

(54) Sustainable 2-Alkyltetrahydrofuran Fuels for Compression Ignition Engines

(71) Applicant: The United States of America, as represented by the Secretary of the Navy, Arlington, VA (US)

(72) Inventor: Matthew C. Davis, Ridgecrest, CA (US)

(21) Appl. No.: 18/952,054

(22) Filed: Nov. 19, 2024

(51) Int. Cl. C10L 1/185; C07D 307/06; C10L 1/02

(52) U.S. Cl. CPC C10L 1/1855; C07D 307/06; C10L 1/026; C10L 2200/04; C10L 2200/0476; C10L 2290/543

(57) ABSTRACT

The current invention describes a high-throughput catalytic process for the production of diesel fuel from domestic agricultural products, furfuraldehyde and plant fatty acids. In the present process, reacting medium and long chain fatty anhydrides (such as capric, caprylic, lauric and palmitic) with furfuraldehyde by a Perkin condensation produces 2-n-alkenylfurans. In a second step, the 2-n-alkenylfurans are hydrogenated to form 2-n-alkyltetrahydrofurans. Basic fuel property testing (melting point, density, kinematic viscosity, derived cetane number and calorific value) of the 2-n-alkyltetrahydrofurans indicates they are potentially useful as fuels for diesel engines. One mixture composed of 2-octyl- and 2-decyltetrahydrofuran had the best combination of fuel properties including a low melting point (−39° C.), high cetane number (63.1), high flash point (98.2° C.) and low viscosity (2.26 mm2/sec, 40° C.) which compares favorably with specifications for diesel #2 and biodiesel.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### FIELD OF THE INVENTION

The invention describes a method to manufacture sustainable diesel fuel by the Perkin reaction of 2-furaldehyde and fatty acid anhydrides. The products have excellent cetane values suitable for compression ignition engines. This class of materials will help reach the goal of 50% renewable fuels for the Navy's Green Fleet challenge.

### BRIEF DESCRIPTION OF THE DRAWINGS

FIG. 1 is an illustration of a method for the synthesis of sustainable 2-n-alkyltetrahydrofuran fuels, according to embodiments of the invention.

FIG. 2 is a GCMS trace of 2-dodecyltetrahydrofuran, according to embodiments of the invention.

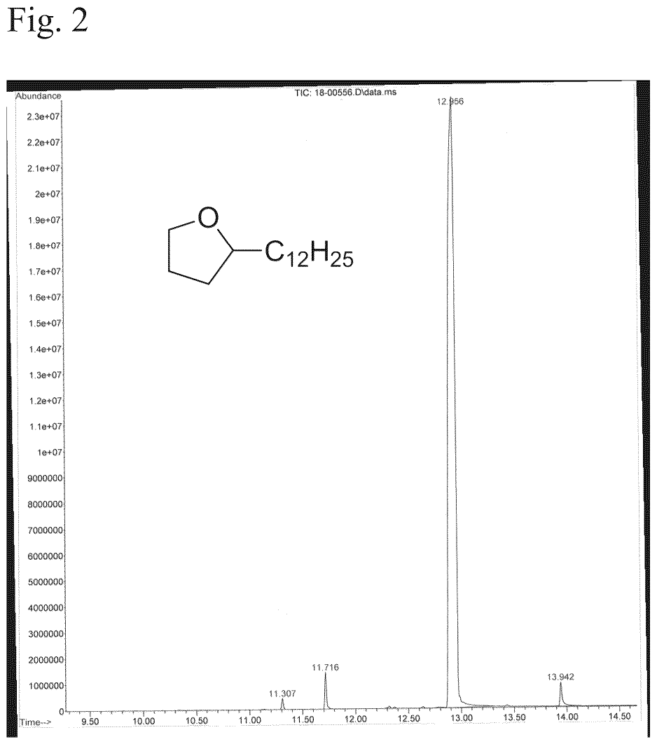

FIG. 3 is a chart of derived cetane numbers for 2-n-alkyltetrahydrofuran sustainable fuels, according to embodiments of the invention . . .

It is to be understood that the foregoing general description and the following detailed description are exemplary and explanatory only and are not to be viewed as being restrictive of the invention, as claimed. Further advantages of this invention will be apparent after a review of the following detailed description of the disclosed embodiments, which are illustrated schematically in the accompanying drawings and in the appended claims.

### Definitions

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Reference throughout the specification to “one example”, “another example”, “an example”, and so forth, means that a particular element (e.g., feature, structure, and/or characteristic) described in connection with the example is included in at least one example described below, and may or may not be present in other examples. In addition, it is to be understood that the described elements for any example may be combined in any suitable manner in the various examples unless the context clearly dictates otherwise.

In describing and claiming the examples described below, the singular forms “a”, “an”, and “the” include plural referents unless the context clearly dictates otherwise.

As used herein, the terms “about” or “approximately” mean within an acceptable range for the particular parameter specified as determined by one of ordinary skill in the art, which will depend in part on how the value is measured or determined, e.g., the limitations of the sample preparation and measurement system. Example of such limitations include preparing the sample in wet versus a dry environment, different instruments, variations in sample height, and differing requirements in signal-to-noise ratios. The use of numerical values in the various ranges specified in this application, unless expressly indicated otherwise, are stated as approximations as though the minimum and maximum values within the stated ranges are both preceded by the word “about” or “approximately”. In this manner, slight variations above and below the stated ranges can be used to achieve substantially the same results as values within the ranges. Also, unless indicated otherwise, the disclosure of these ranges is intended as a continuous range including every value between the minimum and maximum values.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The definitions and understandings of fatty acids, furfuraldehyde, fuels, and related chemistry, are known to those of skill in the art, and such definitions are incorporated herein for the purposes of understanding the general nature of the subject matter of the present application. However, the following detailed description is useful as a further understanding of some of these terms.

### DETAILED DESCRIPTION OF THE EMBODIMENTS OF THE INVENTION

Sustainable Navy operations require access to bio-based turbine and diesel fuels that meet the specifications of conventional jet fuels. The current invention describes a high-throughput catalytic process for the production of sustainable diesel fuel from 2-furaldehyde and fatty acid anhydrides.

Typically, sustainable aviation fuels are prepared from substrates including vegetable oils, syngas, or bioethanol. the resulting fuels are composed primarily of linear alkanes or branched alkanes that don't meet all of the specifications for conventional jet fuel. Another approach to the production of fuels is the production of a molecule or mixture of molecules via fermentation of biomass substrates utilizing a metabolically engineered organism. The resulting molecules can then be converted to fuels through catalytic methods. The current invention describes an efficient catalytic process for the production of diesel fuel from 2-furaldehyde and fatty acid anhydrides. The resulting fuel exhibits cetane numbers far above the minimum for US diesel fuel.

New sources of fuel will become more important in the future as a means to maintain competitive advantage. Fuels that can be made from renewable domestic products will help minimize the country's reliance on foreign sources of petroleum and other energy sources. The US Navy uses vast quantities of hydrocarbon fossil fuels, particularly in the diesel range for locomotion of vehicles (ships, trucks, aircraft). This invention describes a new way to make sustainable diesel fuels from two renewable domestic agricultural products, furfuraldehyde and plant fatty acids, both of which can be obtained from plant crops such as corn, sugarcane or soybean, for example. The furfuraldehyde can be obtained from any of the many bagasse from harvest such as corn husk or cane stalks and the fatty acids are sourced from any kind of vegetable oil that is in surplus. After the reactions, the 2-alkyltetrahydrofuran products have an alkane substituent which is linear and unbranched, which give the molecules good performance in cetane value experiments and are nearly ideal for compression ignition motor combustion. The cetane values for the 2-alkyltetrahydrofurans, where the alkyl radical is four carbons or higher, are greater than the minimum value for US diesel regulations of 40. When the alkyl radical is 12 carbons, made from lauric acid for example, the cetane number was found to be 89. This new method does not require exotic and expensive catalysts, for example, 5% palladium on carbon, a very cheap and reusable catalyst, is used to reduce the 2-alkenylfurans to 2-alkyltetrahydrofurans. Also, the catalytic hydrogenation can be accomplished at low hydrogen pressure (20 to 40 psi) without requiring expensive apparatus (e.g. stainless steel bomb). These new diesel fuels are also naturally very low sulfur, so emissions from burning these fuels will have less impact on the environments (low SOx).

Additionally, embodiments of the invention have the potential to decrease net carbon emissions of various platforms while maintaining optimum performance.

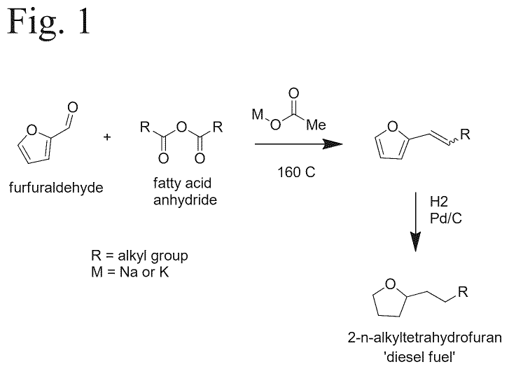

In embodiments, as depicted in FIG. 1, the method comprises:
a) a fatty acid comprising a carbon chain of from 4 to 22 carbon atoms, is allowed to react with excess acetic anhydride at reflux to prepare the fatty acid anhydride;
b) the fatty acid anhydride (carbon chain 4 to 22) is then reacted with furfuraldehyde along with sodium or potassium acetate at a temperature of about 150° to about 200° C. for from about 2 to about 8 hours to yield a 2-alkenylfuran;
c) the 2-alkenylfuran product is distilled from the reaction mixture using reduced pressure of about 60 to about 10 torr; and,
d) the 2-alkenylfuran product is reacted with hydrogen and utilizing a catalyst to prepare the 2-alkyltetrahydrofurans.

The naturally occurring fatty acid (C4 to C22) was reacted with 2.5 equivalents of acetic anhydride for up to about 1 to about 5 hours at a reflux temperature of between about 118° C. and about 140° C. Then the acetic acid and residual acetic anhydride are distilled at atmospheric pressure to obtain the fatty acid anhydride.

In embodiments, the naturally occurring fatty acid (C4 to C22) includes, but is not limited to: Butyric, Valeric, Caproic, Enanthic, Caprylic, Pelargonic, Octanoic, Capric, Undecylic, Lauric, Tridecylic, Myristic, Pentadecylic, Palmitic, Margaric, Stearic, Nonadecylic, Arachidic, Heneicosylic, Behenic, or combinations thereof.

In other embodiments, rather than a single, pure distilled fatty acid, a mixture of two or more fatty acids can also be employed in the carboxylic anhydride reaction to form a mixture of fatty acid anhydrides as well as mixed fatty acid anhydride (R1CO₂COR2), where R1 and R2 are each independently selected from an alkyl of C4 to C22, including but not limited to, Butyric, Valeric, Caproic, Enanthic, Caprylic, Pelargonic, Octanoic, Capric, Undecylic, Lauric, Tridecylic, Myristic, Pentadecylic, Palmitic, Margaric, Stearic, Nonadecylic, Arachidic, Heneicosylic, or Behenic.

The fatty acid anhydride made in the previous step was then reacted with 1 equivalent each of furfuraldehyde and sodium or potassium acetate at a temperature of between about 150° C. to about 200° C. for a period of about 4 to about 6 hours.

The reaction mixture made in the previous reaction was distilled at reduced pressure to obtain the product 2-alkenylfuran species which will vary according to the fatty acid anhydride prepared in the first step. The reaction produces a mixture of both the cis and trans isomers of the 2-alkenylfuran species.

The 2-alkenylfuran product of the previous reaction is then subjected to catalytic hydrogenation at a pressure of from 20 to 40 psi and the catalyst being 5 wt % palladium on carbon, with a catalyst loading of from 5% to 10% the weight of the 2-alkenylfuran. The product of the reduction is the 2-alkyltetrahydrofuran. The hydrogenation reaction can be conducted at moderate temperatures, preferably from ambient to about 50 degrees ° C.

Based upon the starting fatty acid anhydride used in the process described here, 2-alkyltetrahydrofurans can be prepared where the alkyl group can be from normal butyl up to and including normal docosane.

The 2-alkyltetrahydrofurans of this invention can be used to fuel compression ignition motors such as those used in diesel trucks, generators, shipboard motors, etc. The 2-alkyltetrahydrofurans can also be used as a blending agent for typical diesel fuel cuts from petroleum refining to increase cetane number.

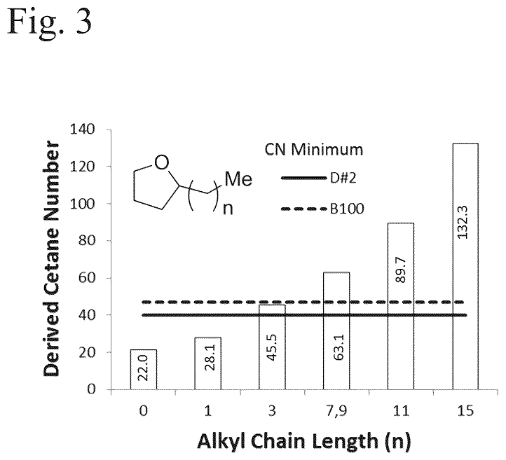

As shown in FIG. 3, the derived cetane number for three examples of 2-alkyltetrahydrofurans of this invention are: 2-hexadecyltetrahydrofuran (cetane number of 132); 2-dodecyltetrahydrofuran (cetane number of 89.7); and a mixture of 2-octyltetrahydrofuran and 2-decyltetrahydrofuran (cetane number of 63.1). The minimum cetane number for US diesel fuel is 40.

In embodiments the catalyst is a platinum on carbon, palladium on carbon, rhodium on carbon, or nickel on carbon catalyst. In preferred embodiments the reaction is conducted without solvent. In other embodiments, suitable solvents useful for reduction include hexanes, tetrahydrofuran, or diethyl ether. Typical catalyst loadings for this reaction range from about 0.001 to about 8 wt % of the 2-alkenylfuran.

### EXAMPLES

### Fatty Acid Anhydride:

### Lauric Anhydride

A round-bottomed flask (3 L) equipped with a magnetic stirring bar and reflux condenser was charged with lauric acid (1.09 kg, 5.4 mol) and Ac₂O (13.5 mol, 1.38 kg, 1.275 L, 2.5 equiv). The mixture was heated with a heating mantle to boiling for 5 h. Afterwards, the reflux condenser was replaced with a distillation head and the reaction mixture was distilled at atmospheric pressure. Once the temperature of the distillate fell below 100° C., the distillation was then conducted at reduced pressure (1.3 kPa) to remove the remaining portion of acetic acid. The distillation pot was allowed to cool to rt during which time the reaction product solidified. No further purification was performed nor was necessary, m.p.: 37-40° C. 1H NMR (300 MHz, CDCl₃, δ ppm): 2.46 (triplet, J=7.6 Hz, 4H), 1.67 (pentet, J=7.4 Hz, 4H), 1.41-1.21 (multiplet, 32H), 0.9 (triplet, J=6.9 Hz, 6H); 13C NMR (75 MHz, CDCl₃, δ ppm): 169.84, 35.52, 32.12, 29.81, 29.79, 29.62, 29.54, 29.42, 29.09, 24.47, 22.89, 14.32. Elemental analysis calculated for C₂₄H₄₆O₃ (382.6): C, 75.34; H, 12.12%. Found: C, 75.35; H, 12.26%.

### 2-Alkenylfuran:

Cis/Trans 2-n-Octenylfuran and Cis/Trans 2-n-Decenylfuran (2/1) Mixture

A two-necked, round-bottomed flask (2 L) equipped with magnetic stirring bar was filled with caprylic/capric anhydride mixture (401 g, 1.39 mol), furfuraldehyde (134 g, 115.5 mL, 1 equiv) and anhydrous KOAc (137 g, 1 equiv). The side neck was equipped with a thermometer to monitor the internal temperature. A reflux condenser was equipped to the other flask neck. The mixture was heated on a stir plate with a heating mantle with the Variac set to 40% power. During the heating up period, it was noticed that some liquid was distilling at ˜100° C. while gas was evolving. The color of the reaction also changed to dark brown during once the mixture reached maximum temperature. After 5 h, evolution of gas had ceased. The heating was shut off to allow the mixture to cool down to ˜100° C. The thermometer was replaced with a glass stopper and the condenser was replaced with a short-path, reduced pressure distillation head. The heating mantle was turned on again and the mixture was distilled (1.3 kPa) to obtain a pale yellow liquid in the receiver (˜100 g). The distillate was partitioned between hexanes (500 mL) and 1M KOH (2 L). After allowing the phases to break, the organic layer was separated and washed with H₂O (1 L) followed by brine (1 L). The organic layer was dried over anhydrous MgSO₄ (10 g) for 20 min, filtered and then the solvent was rotary evaporated. The residue was then distilled at reduced pressure (b.p. 84-94° C., 13 Pa) to give the product as a pale yellow liquid (40 g, 16%). 1H NMR (300 MHz, CDCl₃, δ ppm): 7.43-7.31 (multiplet, 1H), 6.47-5.54 (multiplet, 4H), 2.56-2.12 (multiplet, 2H), 1.60-1.22 (multiplet, 15H), 0.94 (triplet, J=7.2 Hz, 6H). 13C NMR (75 MHz, CDCl₃, δ ppm): 153.41, 141.11, 131.44, 130.27, 118.49, 117.23, 111.02, 108.65, 105.80, 34.69, 32.84, 31.93, 31.78, 31.64, 29.53, 29.33, 29.26, 29.13, 28.92, 25.31, 22.71, 22.69, 22.66, 14.11. Elemental analysis calculated for 1 C₁₂H₁₈O+0.5 C₁₄H₂₂O: C, 81.09; H, 10.39%. Found: C, 79.21; H, 10.14%.

### Catalytic Hydrogenation:

2-n-Dodecyltetrahydrofuran

A mixture of cis/trans 2-n-dodecenylfuran (129.17 g, 550 mmol), 5% Pd on carbon (10 g, 7.7 wt %) and anhydrous THF (100 mL) was loaded into a pressure bottle and hydrogenated (275 kPa) on a Parr shaker apparatus. Uptake of hydrogen was complete after 1.5 h. The mixture was filtered through diatomaceous earth to remove the catalyst and the solvent was rotary evaporated. The crude product was distilled at reduced pressure (b.p. 90-107° C., 13 Pa) to obtain the pure product as a colorless liquid (123 g, 94%). 1H NMR (300 MHz, CDCl₃, δ ppm): 3.93-3.61 (multiplet, 3H), 2.02-1.78 (multiplet, 3H), 1.58-1.16 (multiplet, 25H), 0.88 (triplet, J=7 Hz, 3H). 13C NMR (75 MHz, CDCl₃, δ ppm): 79.43, 67.55, 35.75, 31.92, 31.38, 29.76, 29.67, 29.64, 29.62, 29.60, 29.35, 26.41, 25.70, 22.67, 14.08. Elemental analysis calculated for C₁₆H₃₂O (240): C, 79.93; H, 13.42%. Found: C, 79.97; H, 13.37%.

2-n-Octyltetrahydrofuran and 2-n-Decyltetrahydrofuran (2/1) Mixture

A 2:1 mixture of 2-n-Octenylfuran and 2-n-decenylfuran (129.17 g), 5% Pd on carbon (10 g, 7.7 wt %) and anhydrous THF (100 mL) was loaded into a pressure bottle and hydrogenated (275 kPa) on a Parr shaker apparatus. Uptake of hydrogen was complete after 1.5 hours. The mixture was filtered through diatomaceous earth to remove the catalyst and the solvent was rotary evaporated.

The product was distilled at reduced pressure (13 Pa) to obtain the pure compound as a colorless liquid that had a mild floral odor. 1H NMR (300 MHz, CDCl₃, δ ppm): 3.92-3.57 (multiplet, 3H), 2.0-1.73 (multiplet, 3H), 1.58-1.14 (multiplet, 17H), 0.84 (triplet, J=7 Hz, 3H). 13C NMR (75 MHz, CDCl₃, δ ppm): 79.38, 67.48, 35.71, 31.85, 31.34, 29.73, 29.58, 29.57, 29.55, 26.37, 25.66, 22.61, 14.01. Elemental analysis calculated for 2 C₁₂H₂₄O+1 C₁₄H₂₈O: C, 78.55; H, 13.18%. Found: C, 77.71; H, 12.86%.

2-n-Hexadecyltetrahydrofuran

2-n-hexadecenylfuran (129.17 g), 5% Pd on carbon (10 g, 7.7 wt %) and anhydrous THF (100 mL) was loaded into a pressure bottle and hydrogenated (275 kPa) on a Parr shaker apparatus. Uptake of hydrogen was complete after 1.5 hours. The mixture was filtered through diatomaceous earth to remove the catalyst and the solvent was rotary evaporated.

The product was distilled at reduced pressure (140-150° C., 13 Pa) to obtain the pure compound as a colorless liquid that slowly became a crystalline slush when stored at room temperature overnight. 1H NMR (300 MHz, CDCl₃, δ ppm): 3.91-3.66 (m, 3H), 2.04-1.77 (m, 3H), 1.51-1.22 (m, 31H), 0.89 (triplet, J=7 Hz, 3H). 13C NMR (75 MHz, CDCl3, δ ppm): 79.46, 67.57, 35.76, 31.94, 29.78, 29.71 (overlapping signals), 29.68, 29.64, 29.62, 29.38, 26.43, 25.72, 22.70, 14.11. Elemental analysis calculated for C₂₀H₄₀O (296.5): C, 81.01; H, 13.60%. Found: C, 81.27; H, 13.77%.

2-n-Butyltetrahydrofuran

2-n-butenylfuran (129.17 g), 5% Pd on carbon (10 g, 7.7 wt %) and anhydrous THF (100 mL) was loaded into a pressure bottle and hydrogenated (275 kPa) on a Parr shaker apparatus. Uptake of hydrogen was complete after 1.5 hours. The mixture was filtered through diatomaceous earth to remove the catalyst and the solvent was rotary evaporated.

Reduced pressure distillation (58-62° C., 1.3 kPa) gave this compound as a colorless, mobile liquid with a strong floral odor. 1H NMR (300 MHz, CDCl₃, δ ppm): 3.95-3.51 (multiplet, 3H), 2.02-1.69 (multiplet, 3H), 1.61-1.13 (multiplet, 7H), 0.85 (triplet, J=7.2 Hz, 3H). 13C NMR (75 MHz, CDCl₃, δ ppm): 79.34, 67.47, 35.37, 31.31, 28.52, 25.64, 22.74, 13.95. Elemental analysis calculated for C₈H₁₆O (128): C, 74.94; H, 12.58%. Found: C, 73.27; H, 12.25%.

TABLE 1
Derived Cetane Number (DCN) and ignition delay time
(τ) from ignitional quality test (IQT) apparatus.

| | Molecular | | |
|---|---|---|---|
| | formula | τ (ms) | DCN |
| 2-octylTHF/ | C_{12.6}H_{25.3}O | 3.18 | 63.1 |
| 2-decylTHF | | | |
| 2-dodecylTHF | C₁₆H₃₂O | 2.47 | 89.7 |
| 2-hexadecylTHF | C₂₀H₄₀O | 2.03 | 132.3 |

It is to be understood that the foregoing is exemplary and explanatory only and are not to be viewed as being restrictive of the invention, as claimed. Further advantages of this invention will be apparent after a review of the following detailed description of the disclosed embodiments, which are illustrated schematically in the accompanying drawings and in the appended claims.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

What is claimed is:

1. A method for manufacturing 2-alkyltetrahydrofuran diesel, comprising:
reacting one or more fatty acids with a stoichiometric excess of acetic anhydride at reflux to prepare a fatty acid anhydride;
reacting a reaction mixture comprising the fatty acid anhydride with furfuraldehyde along with sodium or potassium acetate to produce a 2-alkenylfuran product;
distilling the 2-alkenylfuran product from the reaction mixture; and,
reacting the 2-alkenylfuran product with hydrogen to produce 2-alkyltetrahydrofurans.

2. The method according to claim 1, wherein the one or more fatty acids is comprised of carbon chains of 2 to 22 carbon atoms.

3. The method according to claim 1, wherein the fatty acid anhydride is reacted with furfuraldehyde at a temperature from about 150° C. to about 200° C.

4. The fatty acid anhydride is reacted with furfuraldehyde for from about 2 to about 8 hours.

5. The method according to claim 1, wherein the one or more fatty acids are selected from the group comprising Butyric, Valeric, Caproic, Enanthic, Caprylic, Pelargonic, Octanoic, Capric, Undecylic, Lauric, Tridecylic, Myristic, Pentadecylic, Palmitic, Margaric, Stearic, Nonadecylic, Arachidic, Heneicosylic, and combinations thereof.

6. The method according to claim 1, wherein the 2-alkenylfuran product is distilled from the reaction mixture using reduced pressure of about 60 to about 10 torr.

7. The method according to claim 1, wherein the 2-alkenylfuran product reaction with hydrogen is catalyzed using a platinum on carbon, palladium on carbon, rhodium on carbon, or nickel on carbon catalyst.

8. The method according to claim 7, wherein the catalyst loading is from about 0.001 to about 8 wt %.

9. The method according to claim 1, wherein the 2-alkyltetrahydrofurans have a cetane number higher than about 40.

10. The method according to claim 9, wherein the 2-alkyltetrahydrofurans have a cetane number higher than about 80.

11. The method of claim 1, wherein the 2-alkenylfuran products are comprised of a combination of cis and trans isomers.

12. The method of claim 11, wherein the 2-alkenylfuran products are comprised of: 2-butenylfuran, 2-pentenylfuran, 2-hexenylfuran, 2-heptenylfuran, 2-octenylfuran, 2-nonenylfuran, 2-decenylfuran, 2-undecenylfuran, 2-dodecenylfuran, 2-tridecenylfuran, 2-tetradecenylfuran, 2-pentadecenylfuran, 2-hexadecenylfuran, 2-heptadecenylfuran, 2-octadecenylfuran, 2-nonadecenylfuran, 2-icosenylfuran, 2-heneicosenylfuran, or mixtures thereof.

13. The method of claim 1, wherein the 2-alkyltetrahydrofurans are comprised of: 2-butyltetrahydrofuran, 2-pentyltetrahydrofuran, 2-hexyltetrahydrofuran, 2-heptyltetrahydrofuran, 2-octyltetrahydrofuran, 2-nonyltetrahydrofuran, 2-decyltetrahydrofuran, 2-undecyltetrahydrofuran, 2-dodecyltetrahydrofuran, 2-tridecyltetrahydrofuran, 2-tetradecyltetrahydrofuran, 2-pentadecyltetrahydrofuran, 2-hexadecyltetrahydrofuran, 2-heptadecyltetrahydrofuran, 2-octadecyltetrahydrofuran, 2-nonadecyltetrahydrofuran, 2-icosyltetrahydrofuran, 2-heneicosyltetrahydrofuran, or mixtures thereof.
